(19) United States
(12) Patent Application Publication
(10) Pub. No.: US 2012/0172877 A1
(43) Pub. Date: Jul. 5, 2012

(54) SURGICAL TOOL COUPLING

(75) Inventors: Phillip A. Ryan, Memphis, TN (US); Kevin C. Edwards, Olive Branch, MS (US)

(73) Assignee: GYRUS ENT, L.L.C., BARTLETT, TN (US)

(21) Appl. No.: 12/984,296

(22) Filed: Jan. 4, 2011

(51) Int. Cl. A61B 17/00; A61B 17/32

(52) U.S. Cl. 606/79; 606/170

(57) ABSTRACT

A surgical instrument includes distal and proximal tube sections and a coupling that couples the distal tube section to the proximal tube section. The coupling includes first and second connector portions coupled to one of the distal tube section and the proximal tube section. The first and second connector portions are selectively engageable and disengageable with respect to each other and each include selectively engageable fixing members. The distal and proximal tube sections are rotationally fixed to each other when the fixing members of the second connector portion are engaged with the fixing members of the first connector portion. The distal and proximal tube sections can rotate relative to each other when the fixing members of the second connector portion are disengaged from the fixing members of the first connector portion.

### BACKGROUND

This disclosure relates to improved surgical instruments, couplings for attaching surgical instrument tube sections together and methods of performing surgical procedures with the surgical instruments. In particular, the disclosure relates to surgical instruments usable, for example, to shave, cut, resect, abrade and/or remove, tissue, bone and/or other bodily materials, in which the instrument includes a coupling to adjustably, rotatably couple outer tube sections of the surgical instrument. The invention, however, is not limited to cutting instruments but is applicable to various surgical instruments, such as those used for suctioning, cauterizing, grasping or guiding a light source, in which instrument sections are rotatably coupled to each other.

Surgical apparatus used to shave, cut, abrade and/or remove tissue, bone and/or other bodily materials are known. Such surgical apparatus can include a cutting surface, such as a rotating blade, disposed on an elongated inner tube that is rotated within an elongated outer tube having a cutting window. The inner and outer tubes together form a surgical cutting blade. In general, the elongated outer tube includes a distal end defining an opening or cutting window that exposes the cutting surface of the inner tube (at the distal end of the inner tube) to tissue, bone and/or any other bodily materials. A powered handpiece is used to rotate the inner tube with respect to the outer tube while an outer tube hub (rigidly connected to the proximal end of the outer tube) is removably, rigidly fixed against rotation and axial movement with respect to the handpiece and an inner tube hub (rigidly connected to the proximal end of the inner tube) is held in place by the powered handpiece and driven by the handpiece to rotate.

These surgical apparatus are also known to have bends at either, or both, the distal and proximal ends of the outer tube in order to orient the cutting window in a particular plane. Thus, the inner tube, disposed inside the outer tube, includes flexible portions adjacent the bent portions of the outer tube that allow for the inner tube to be inserted in the outer tube and rotate within the outer tube once bent.

During surgery, it may be necessary or useful to reorient a tip and/or bend of the surgical cutting blade such that the blade tip is in a different position and/or bend is in a different plane. Currently, in order to reorient the tip of the blade, a surgeon must alter the position of his/her hand or switch hands in order to reposition the tip as well as the cutting window of the outer tube or the plane in which the tip is disposed.

U.S. Pat. No. 7,276,074 to Adams et al. (hereinafter “Adams”) is representative of an angled tissue cutting instrument including a bend at the proximal portion of the outer member with respect to the forward end of the body. The outer member includes a main body portion and a distal tip that includes the cutting window. In order to reorient the cutting window to a selected directional position, the distal tip is detachable from the main body portion and can be rotated such that the cutting window faces a new direction before reattachment to the main body portion.

However, the above surgical instruments result in a number of disadvantages during surgery including the need for a surgeon to alter his/her hand position. For instance, with respect to Adams, the surgeon must move his/her hands from their first natural position to change directions of the bend at the distal end of the outer member, or to remove the distal tip from the main body position in order to rotate and then reattach the detached distal tip, Moreover, the surgeon must completely remove the surgical instrument from the patient to change the orientation of the tip.

### SUMMARY

The preference of a user (i.e., the preferences of the surgeon performing an operation) as well as the demands of surgery dictate infinite requirements for the orientation of the cutting window as well as the plane in which the bend of the outer tube lies. Currently, accommodating such user preferences and surgery requirements during surgical procedures requires the orientation of the user's hand position or the removal, rotation and reattachment of portions of the outer tube.

It would be advantageous to provide an arrangement that would allow the surgeon to change the plane in which the distal end of the outer tube lies as well as the orientation of the cutting window without having to move his/her hand from a desired position. According to some aspects of the invention, a coupling is provided on the outer tube that allows a user to change the plane in which the distal end of the outer tube is disposed (by rotating the distal tube section) with just a slight movement of the thumb, for example.

In accordance with one aspect of the invention, a surgical instrument may include a distal tube section having a bend therein, a proximal tube section, and a coupling that couples the distal tube section to the proximal tube section. The coupling may include a first connector portion coupled to a first one of the distal tube section and the proximal tube section, and a second connector portion coupled to a second one of the distal tube section and the proximal tube section. The first and second connector portions may be selectively engageable and disengageable with respect to each other. The first and second connector portions may each include selectively engageable fixing members such that the distal and proximal tube sections are rotationally fixed to each other when the fixing members of the second connector portion are engaged with the fixing members of the first connector portion, and the distal and proximal tube sections can rotate relative to each other when the fixing members of the second connector portion are disengaged from the fixing members of the first connector portion.

In some embodiments in which the surgical instrument is a cutting instrument, the coupling is coupled to a proximal end of the distal tube section, the distal tube section includes a cutting window at its distal end, and the bend of the distal tube section is disposed between the distal and proximal ends of the distal tube section.

In some embodiments, the proximal tube section includes a bend. The bend of the distal tube section and the bend of the proximal tube section can be disposed in a common plane or in different planes by rotating the first and second connector portions relative to each other.

In some embodiments, the first connector portion includes a fixed sleeve. The second connector portion includes a sliding sleeve, and the sliding sleeve is movable toward and away from the fixed sleeve. The distal and proximal tube sections are rotationally fixed to each other when the sliding sleeve is moved toward the fixed sleeve such that the fixing members are engaged with each other. The distal and proximal tube sections can rotate relative to each other when the sliding sleeve is moved away from the fixed sleeve such that the fixing members are disengaged from each other.

In some embodiments, the coupling includes a rotating sleeve having a first end that is fixed to the distal tube section, and a second end that is rotatably coupled to the fixed sleeve.

In some embodiments, the rotating sleeve includes a pin that extends into a slot of the sliding sleeve to prevent the rotating and sliding sleeves from rotating relative to each other.

In some embodiments, the rotating sleeve is disposed inside of the fixed and sliding sleeves such that inner surfaces of the fixed and rotating sleeves, together with inner surfaces of the distal and proximal tube sections, form an internal passage of the surgical instrument that is maintained when the fixed and sliding sleeves are rotated relative to each other.

In some embodiments, a spring is disposed between the rotating and sliding sleeves, and the spring biases the sliding sleeve toward the fixed sleeve such that the fixing members of the fixed and sliding sleeves engage with each other.

In some embodiments, the rotating sleeve is coupled to the proximal tube section and the sliding sleeve is coupled to the rotating sleeve.

Another aspect of the invention relates to a coupling for attaching tube sections of a surgical instrument to each other. The coupling may include a fixed sleeve having first and second ends such that the first end is fixedly attached to a first one of the tube sections. The coupling may also include a rotating sleeve having first and second ends such that the first end is fixedly attached to a second one of the tube sections and the second end is rotatably attached to the fixed sleeve. The coupling may include a sliding sleeve disposed over an outer surface of the rotating sleeve and being slidable relative to the fixed and rotating sleeves between a locked position at which the sliding sleeve is closest to the fixed sleeve and an unlocked position at which the sliding sleeve is disposed away from the fixed sleeve. The coupling sleeve may also include engageable fixing members provided on the second end of the fixed sleeve and on an end of the sliding sleeve that is engageable with the fixed sleeve. When the sliding sleeve is moved to the locked position, the fixing members are engaged with each other to prevent relative rotation between the fixed and rotating sleeves. When the sliding sleeve is moved to the unlocked position, the fixing members are disengaged from each other such that the fixed and rotating sleeves can rotate relative to each other.

In some embodiments, the second end of the rotating sleeve includes a flanged portion that is received within an accommodation portion at the second end of the fixed sleeve.

In some embodiments, the accommodation portion is configured to permit rotation and restrict axial movement of the rotating sleeve relative to the fixed sleeve.

In some embodiments, a spring is disposed between the rotating sleeve and the sliding sleeve, and the spring biases the sliding sleeve toward the locked position.

In some embodiments, the rotating sleeve is disposed inside of at least part of the fixed and sliding sleeves such that inner surfaces of the fixed and rotating sleeves, together with inner surfaces of the surgical instrument tube sections, form an internal passage of the surgical instrument that is maintained (i.e., not separated) when the fixed and rotating sleeves are rotated relative to each other.

According to some aspects of the invention, a method of performing a surgical procedure with the surgical instrument includes inserting at least a portion of the distal tube section of the surgical instrument into a passage of a patient. While the surgical instrument is inserted into the patient or the surgical instrument is external to the patient, the coupling is moved to an unlocked position at which the fixing members of the fixed and sliding sleeves are disengaged from each other. While in the unlocked position, the distal and proximal tube sections are rotated relative to each other. After the rotating, the coupling is moved to a locked position at which the fixing members of the fixed and sliding sleeves are engaged with each other such that the distal and proximal tube sections cannot be rotated relative to each other. Thus, the locking and unlocking of the coupling can be accomplished while the surgical instrument is inserted into a patient or while the surgical instrument is external to the patient.

In some embodiments, a fluid passage through an entirety of the surgical instrument is maintained intact while the coupling is in the unlocked position.

In some embodiments, the coupling includes (a) a fixed sleeve having first and second ends, the first end being fixedly attached to the proximal tube section, (b) a rotating sleeve having first and second ends, the first end being fixedly attached to the distal tube section, the second end rotatably attached to the fixed sleeve, (c) a sliding sleeve disposed over an outer surface of the rotating sleeve and being slidable relative to the fixed and rotating sleeves between the locked position at which the sliding sleeve is closest to the fixed sleeve and the unlocked position at which the sliding sleeve is disposed away from the fixed sleeve, and (d) engageable fixing members provided on the second end of the fixed sleeve and on an end of the sliding sleeve that is engageable with the fixed sleeve. The coupling is moved to the unlocked position by sliding the sliding sleeve over the rotating sleeve and away from the fixed sleeve such that the fixing members on the fixed and sliding sleeves are disengaged from each other. The rotating is performed by rotating the sliding and rotating sleeves relative to the fixed sleeve. The coupling is moved to the locked position by sliding the sliding sleeve over the rotating sleeve and toward the fixed sleeve such that the fixing members on the fixed and sliding sleeves are engaged with each other.

### BRIEF DESCRIPTION OF THE DRAWINGS

Various exemplary embodiments of a surgical instrument to which aspects of the invention are applied will be described in detail with reference to the following drawings in which:

FIG. 1 is a perspective view of a powered surgical tool system that incorporates a surgical instrument, a controller, a power source, a fluid source and a suction source;

FIG. 2 is a side view of an exemplary embodiment of the surgical instrument incorporating a coupling;

FIG. 3 is a side view of FIG. 2 after rotation of the coupling;

FIG. 4 is an enlarged side view of a coupling according to one embodiment;

FIG. 5 is a cross-sectional view of FIG. 4;

FIG. 6A is an enlarged perspective view of an exemplary embodiment of the coupling when force is applied to separate the coupling; FIG. 6B is an enlarged perspective view of an exemplary embodiment of the coupling when the coupling is separated and the outer tube is rotated; FIG. 6C is an enlarged perspective view of an exemplary embodiment of the coupling after rotation of the outer tube and release of the force applied to separate the coupling;

FIG. 7 is an enlarged side view of a coupling according to another embodiment;

FIG. 8 is a cross-sectional view of FIG. 7;

FIG. 9A is an enlarged side view of an exemplary embodiment of the coupling when force is applied to separate the coupling; FIG. 9B is an enlarged perspective view of an exemplary embodiment of the coupling when the coupling is separated and the outer tube is rotated; FIG. 9C is an enlarged perspective view of an exemplary embodiment of the coupling after rotation of the outer tube and release of the force applied to separate the coupling; and

FIGS. 10A-B are side views of the surgical instrument of FIG. 2 during a surgical procedure in which it is deployed in the passage of a patient.

### DETAILED DESCRIPTION OF EMBODIMENTS

Exemplary embodiments of surgical instruments to which aspects of the invention are applied are described below with reference to the figures in the context of human surgery, such as ear, nose and throat surgery, and in particular larynx surgery as well as sinus and nasopharynx surgery. However, the invention is applicable to any instrument, and in particular any surgical instrument in which two tube sections are adjustably, rotatably coupled to each other.

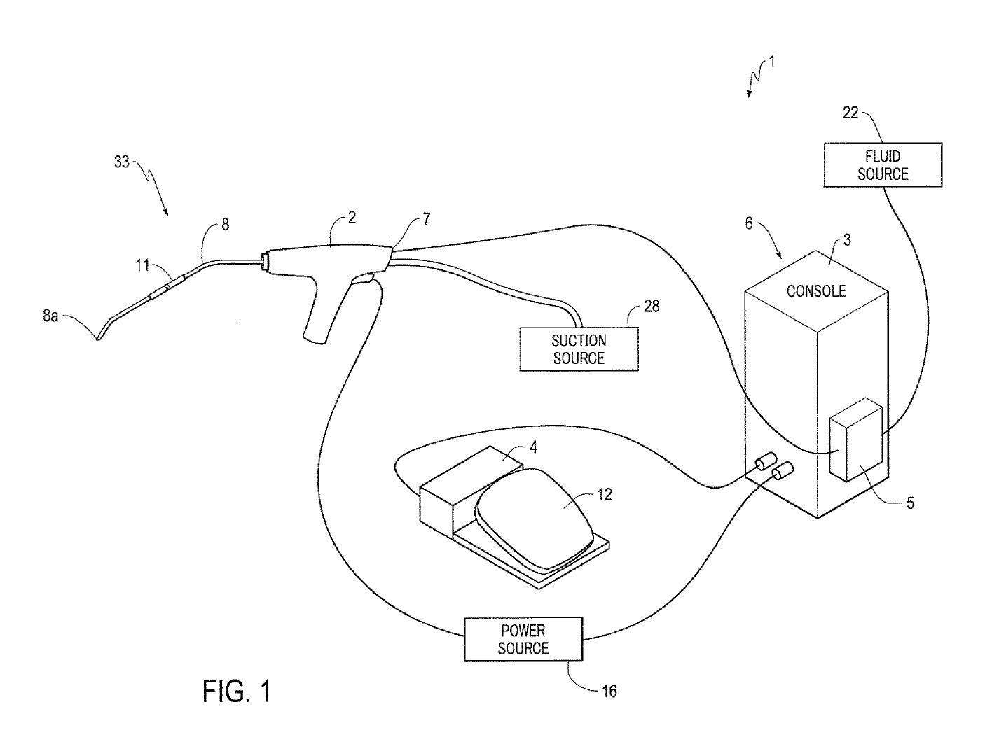

FIG. 1 is a schematic of a powered surgical instrument system. Except for the surgical instrument, to be described hereafter, the system may be in accordance with the system disclosed in U.S. Pat. No. 7,247,161, the disclosure of which is incorporated herein by reference in its entirety. As shown in FIG. 1, the powered tool system 1 includes a handle 2, a footswitch 4 (with pedal 12), power source 16, fluid (liquid and/or gas) source 22, suction source 28, a controller 6, console 3, fluid pump 5 and a fluid inlet/irrigation outlet 7. The handle 2 is connected, at its distal end, to a surgical instrument 8. The surgical instrument includes a cutting instrument 8a at its distal end that is used to cut, shave, resect and/or abrade tissue, bone and/or other bodily materials.

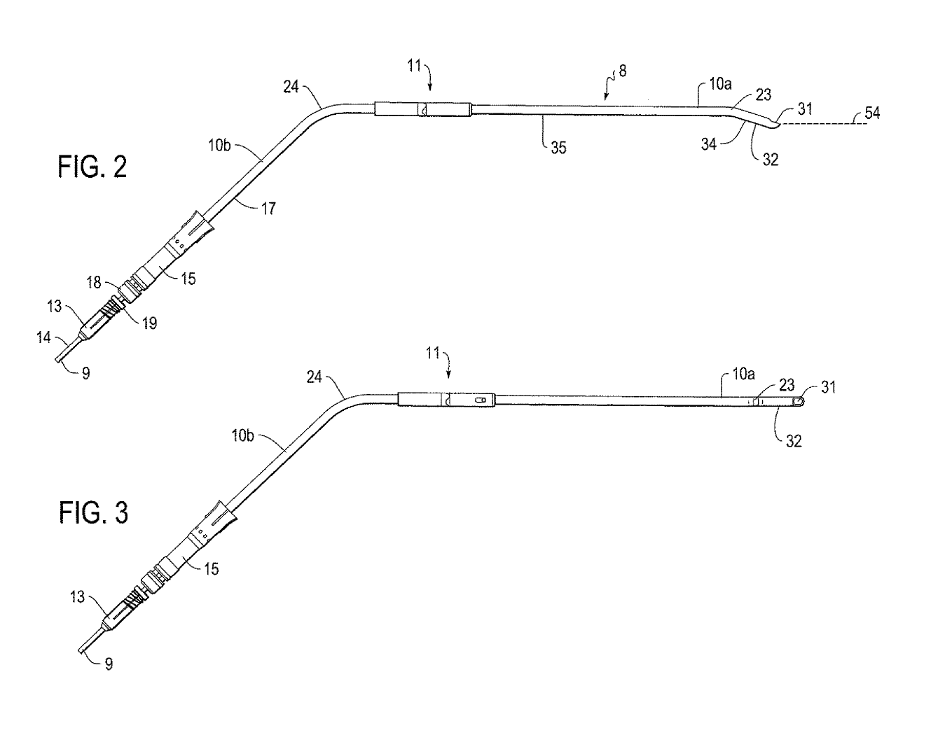

FIG. 2 illustrates a side view of an exemplary embodiment of the surgical instrument 8, in accordance with aspects of the invention. The instrument 8 incorporates an inner tube 9, an outer tube 10 and a coupling 11 disposed on the outer tube 10. In this exemplary embodiment, an inner tube hub 13 is formed on the second end 14 of the inner tube 9 and an outer tube hub 15 is formed on the second end 17 of the outer tube 10. The inner tube 9 (FIG. 2) is inserted into a fluid passage 20 (FIG. 5) formed within the outer tube 10 so that the inner tube 9 is co-axially disposed within the outer tube 10 until the top portion 19 of the inner tube hub 13 contacts or nearly contacts the bottom portion 18 of the outer tube hub 15. The outer tube 10 has a larger diameter than the inner tube 9, thus allowing for insertion of the inner tube 9 within the outer tube 10. However, it should be appreciated that the inner and outer tubes 9, 10 will be pre-assembled prior to delivery to the customer. Thus, a customer will most likely not be inserting the inner tube into the outer tube. Instead, the customer will merely be utilizing the coupling disposed on the outer tube to rotate a section of the outer tube and orient the distal bend, for example, in a different plane than previously disposed.

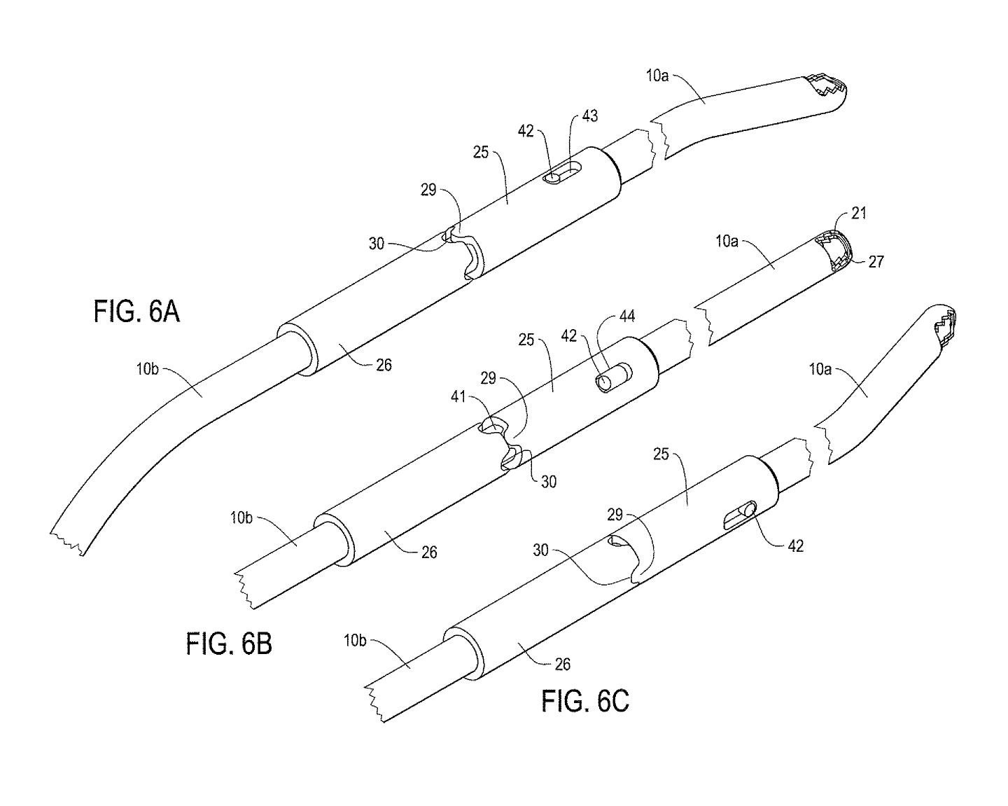

The inner tube 9 includes a fluid/bodily materials removal passage (not shown) that extends the length of the inner tube 9. The inner tube 9 also includes a cutting surface 21 (FIG. 6B) at its first end 27 (FIG. 6B) while the outer tube 10 includes a cutting window 31 at its first end 32. The inner tube 9 is co-axially disposed within the outer tube 10 such that the cutting surface 21 is exposed at the cutting window 31. The cutting surface 21 disposed within the cutting window 31 forms a cutting instrument 8a, which cuts by rotating the inner tube 9 within the outer tube 10, The inner tube 9, inner tube hub 13, outer tube 10, outer tube hub 15 and coupling form a main unit 33 (FIG. 1). The inner and outer tube hubs 13, 15 couple the inner and outer tubes 9, 10, respectively, to the handle 2 such that the inner tube 9 is rotatable relative to the outer tube 10 and the handle 2. The proximal end of the outer tube does not rotate relative to the handle 2 once it is coupled to the handle.

In an exemplary embodiment, the outer tube 10 has a distal tube section 10a and a proximal tube section 10b. For example, the distal tube section 10a has a first bend 23 and the proximal tube section 10b has a second bend 24. The coupling 11, disposed between the first bend 23 and the second bend 24, couples the distal tube section 10a to the proximal tube section 10b. However, the placement of the coupling can be anywhere along the outer tube but not distal of the first bend 23. Additionally, the outer tube may be straight, have a single bend (in either the distal or proximal tube section), or have more than two bends with a coupling disposed somewhere along the length of the outer tube between the distal and proximal ends of the outer tube 10.

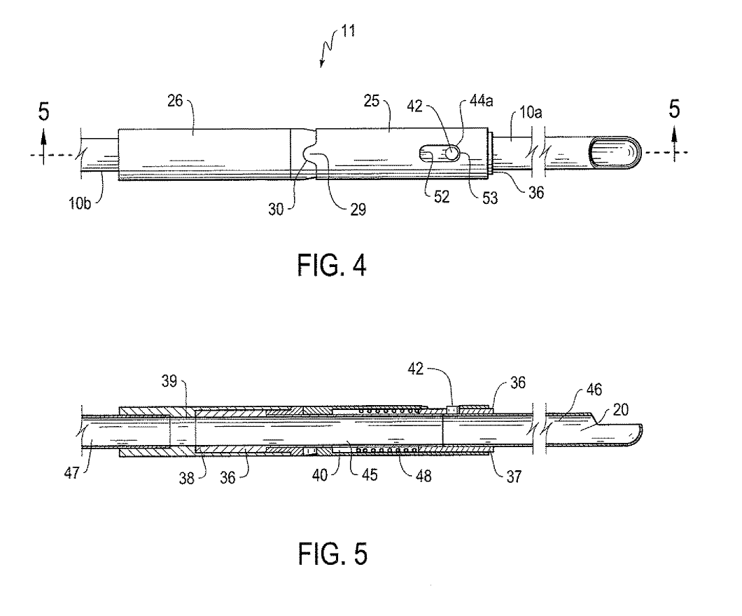

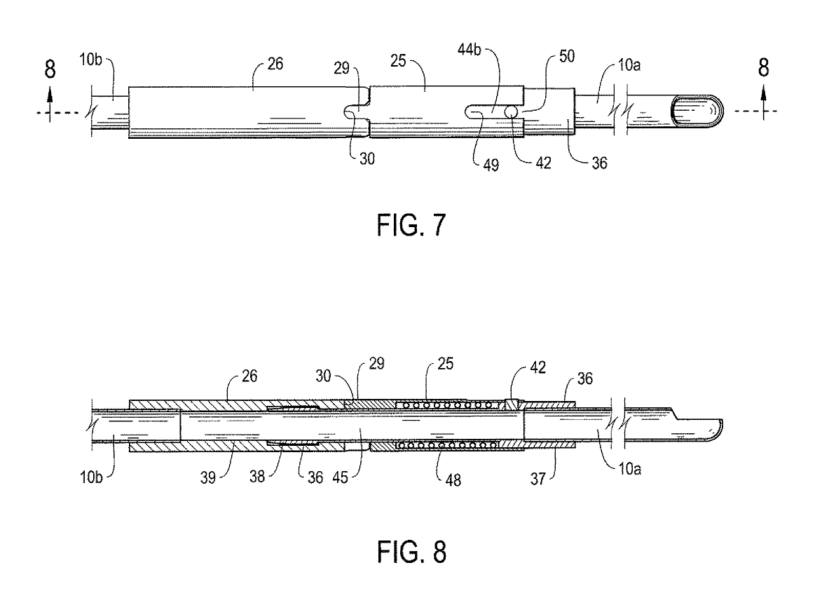

FIGS. 4 and 7 illustrate an enlarged side view of the coupling 11 of first and second exemplary embodiments. The distal tube section 10a is coupled to a rotating sleeve 36 and the proximal tube section 10b is coupled to a fixed sleeve 26, or vice versa. A sliding sleeve 25 and the fixed sleeve 26 are selectively engageable and disengageable with respect to each other. The sliding sleeve 25 includes a selectively engageable fixing member 29 and the fixed sleeve 26 includes selectively engageable fixing members 30. The fixing members 29 and 30 are undulating surfaces forming interlocking teeth and recesses. When the fixing members 30 of the fixed sleeve 26 are engaged with the fixing member 29 of the sliding sleeve 25, the distal tube section 10a is rotationally fixed to the proximal tube section 10b. When the fixing members 30 of the fixed sleeve 26 are disengaged from the fixing member 29 of the sliding sleeve 25, the distal tube section 10a can rotate relative to the proximal tube section 10b. Thus, the first bend 23 and second bend 24 can be disposed in a common plane (FIG. 2) or in different planes (FIG. 3) by rotation of the sliding sleeve 25 (and the attached distal tube section 10a) relative to the fixed sleeve 26 (and the attached proximal tube section 10b). In FIG. 2, bends 24 and 23 are orientated such that tube sections 10b and 10a are in the plane of the page. In FIG. 3, tube section 10b remains in the plane of the page but tube section 10a has been rotated 90° so that is in a plane perpendicular to the page.

The first bend 23 is disposed between a distal end 34 of the distal tube section 10a and a proximal end 35 of the distal tube section 10a. The coupling 11 is coupled to the proximal end 35 of the distal tube section 10a.

In an exemplary embodiment, for example, the fixed sleeve 26 includes fixing members 30. The fixing members 30 can be a plurality of receiving portions 30. The sliding sleeve 25 can include fixing member 29. The fixing member 29 can be a single or a plurality of receiving protrusions 29. The placement and shapes of the fixing members 29, 30 are not limited to the embodiment described above and illustrated herein. For example, a plurality of fixing members 29 can be used with a plurality of fixing members 30 and a single fixing member 29 can be used with a plurality of fixing members 30.

In an exemplary embodiment, for example, the fixing members 30 include four receiving portions. The four receiving portions 30 are formed on the fixed sleeve 26 in intervals of 90° about the longitudinal axis 54 of the surgical instrument 8. However, the fixing members 30 are not limited to this configuration. The fixing members 30 could include more or less than four receiving portions 30. Additionally, the fixing member 29 includes at least one protrusion. The fixing member 29 could be formed on the sliding sleeve 25. However, the fixing member 29 is not limited to this configuration. The placement and shapes of the fixing members 29, 30 are not limited to the embodiment described above and illustrated herein.

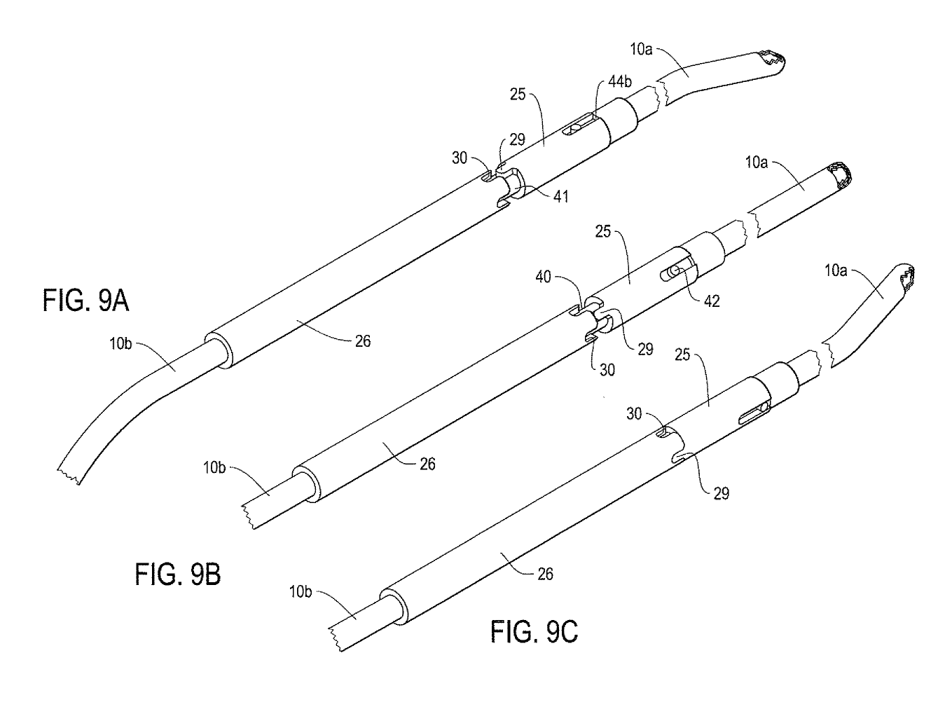

In an exemplary embodiment (FIGS. 5 and 8), for example, the sliding sleeve 25 can move towards and away from the fixed sleeve 26 when force is applied in an axial direction to the coupling 11 (FIGS. 6A and 9A). The movement of the sliding sleeve 25 away from the fixed sleeve 26 disengages the fixing members 29, 30 from each other and allows for rotation of the distal tube section 10a relative to the proximal tube section 10b (FIGS. 6B and 9B). When force is not applied in an axial direction to the coupling 11, the distal tube section 10a and the proximal tube section 10b are rotationally fixed to each other such that the fixing members 29, 30 are engaged with each other (FIGS. 6C and 9C).

As illustrated in FIGS. 5 and 8, the coupling 11 further includes the rotating sleeve 36 having a first end 37 and a second end 38. The second end 38 of the rotating sleeve 36 is rotatably coupled to the fixed sleeve 26 and the first end 37 of the rotating sleeve 36 is fixed to the sliding sleeve 25 and the distal tube section 10a. FIG. 5 illustrates this exemplary embodiment in which the second end 38 of the rotating sleeve 36 is rotatably coupled to the fixed sleeve 26 and the first end 37 of the rotating sleeve 36 is fixed to the proximal end 35 of the distal tube section 10a.

FIGS. 5 and 8 illustrate a cross-sectional view of the couplings of FIGS. 4 and 7, respectively. The rotating sleeve 36 is disposed inside of the sliding sleeve 25 and the fixed sleeve 26 such that the rotating sleeve 36 is below an inner surface 39 of the fixed sleeve 26 and an inner surface 40 of the sliding sleeve 25. An inner surface 45 of the rotating sleeve 36, inner surface 46 of the distal tube section 10a, inner surface 39 of the fixed sleeve 26, and inner surface 47 of the proximal tube section 10b form the fluid passage 20 through which the inner tube 9 extends (the inner tube 9 is not illustrated in FIGS. 5 and 8 as extending through the fluid passage 20) The fluid passage 20 is maintained upon movement (i.e., relative rotation) of the fixed and sliding sleeves (26 and 25) with respect to each other. When pressure is applied to move the sliding sleeve 25 in an axial direction along a length of the outer tube 10, an outer surface 41 of the rotating sleeve 36 is exposed (FIGS. 6A-6B and 9A-9B); however, the fluid passage 20 remains intact.

The rotating sleeve 36 may further include a protrusion 42 that extends outwards from an upper surface 43 of the rotating sleeve 36 and extends into a slot 44 formed in the sliding sleeve 25. The protrusion 42 may be a pin. The pin 42 extending through the slot 44 prevents rotation of the rotating sleeve 36 with respect to the sliding sleeve 25. Thus, the rotating sleeve 36 and sliding sleeve 25 cannot rotate relative to each other but can slide in an axial direction of the coupling 11 relative to each other. A spring 48 is disposed between the rotating sleeve 36 and the sliding sleeve 25 (FIGS. 5 and 8). The spring 48 biases the sliding sleeve 25 toward the fixed sleeve 26 such that the fixing members 29, 30 engage with each other.

FIGS. 4 and 5 illustrate an exemplary embodiment of the pin 42 extending through the slot 44 that is a closed-ended slot 44a. When an axial force is applied to the coupling, the fixing members 29, 30 are disengaged from each other. Upon disengagement, the sliding sleeve 25 moves away from the fixed sleeve 26. The spring 48 is also forced to compress into a compressed position (FIG. 6A). The pin 42 moves along the closed-ended slot 44a for a distance but, upon contact with, or upon nearly contacting, a first end 52 of the closed-ended slot 44a, movement of the pin 42 within the slot 44a is stopped as well as the compression movement of the spring 48 and the movement of the sliding sleeve 25 away from the fixed sleeve 26. Upon disengagement of the fixing members 29, 30, the distal tube section 10a can be rotated relative to the proximal tube section 10b. The rotation of the distal tube section 10a rotates the first bend 23 of the outer tube 10 to lie in a different plane than the second bend 24 of the outer tube (FIGS. 2, 3, and 6B) or in the same plane with a different cutting window orientation, if rotated 180°. The axial force can then be released such that the fixing members 29, 30 reengage. Upon reengagement of the fixing members 29, 30, the spring 48 is released from its compressed position to return to its original semi-compressed position (FIG. 6C) and the pin 42 comes in contact with, or nearly contacts, the second end 53 of the slot 44a (FIG. 4).

FIGS. 7 and 8 illustrate an exemplary embodiment of the pin 42 extending through the slot 44 that is an open-ended slot 44b. The open-ended slot 44b is a slot 44 formed in the sliding sleeve 25 where a first end 49 of the slot 44b is closed while the second end 50 of the slot 44b is open. The slot 44b functions in a similar manner as the slot 44a (FIGS. 9A-C). However, the second end 50 of the slot 44b is open. Thus, in the locked position (i.e., the fixing members 29, 30 are engaged), the spring 48 is extended to an uncompressed position allowing the pin 42 to be positioned at a position in the slot 44b that corresponds to the uncompressed position of the spring 48.

As discussed above, FIGS. 4 and 7 illustrate first and second embodiments. The embodiment of FIG. 4 illustrates a pin-in-slot configuration wherein the slot is closed at both ends. On the other hand, the embodiment of FIG. 7 illustrates a pin-in-slot configuration wherein the slot is closed at a one end and open at the other end. In addition, the fixed sleeve 26 and the rotating sleeve 36 in the FIG. 4/5 embodiment are made from two pieces (shown in FIG. 5) that are welded or otherwise affixed to each other, depending on constituent materials used to assemble the coupling, together after the enlarged portion of the rotating sleeve 36 is inserted into the fixed sleeve 26. In the FIG. 7/8 embodiment, the rotating sleeve 36 is a continuous cylinder throughout its length. In the embodiment of FIGS. 7 and 8, the fixed sleeve 26 is one piece (shown in FIG. 8) and the enlarged portion of the rotating sleeve 36 includes longitudinal slots (not shown) that allow the enlarged end of the rotating sleeve to radially compress while it is snapped into the fixed sleeve 26. Thus, FIG. 7 illustrates an embodiment requiring a fewer number of pieces than the embodiment of FIG. 4. On the other hand, the embodiment of FIG. 7 is not as rigid and thus, not as stable as the embodiment of FIG. 4.

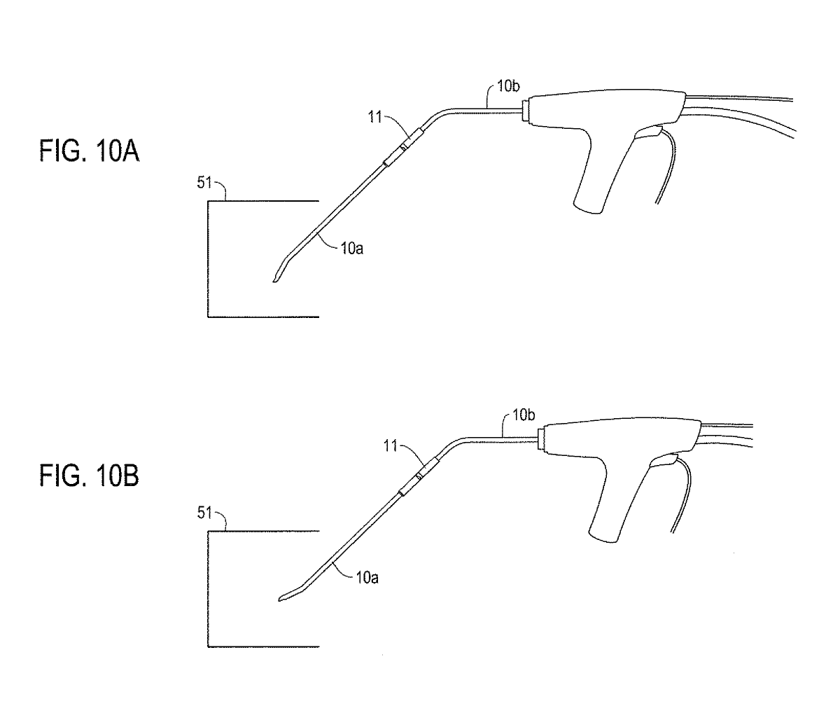

The surgical instrument 8 of the above described embodiments can be used to perform surgical procedures. As illustrated in FIG. 10A, a portion of a least the distal tube section 10a of the surgical instrument 8 is inserted into a passage 51 of a patient so as to perform surgery using the surgical instrument 8. While maintaining the surgical instrument in the passage 51 of the patient, the coupling can be moved to an unlocked position by applying force in an axial direction along the length of the outer tube 10. In the unlock position, the fixing members 29, 30 of the first and second connector portions 25, 26 are disengaged from each other (FIGS. 6A and 9A). The distal tube section 10a is then rotated relative to the proximal tube section 10b (FIGS. 6B and 9B). Upon completion of the rotation, axial force is released and the coupling is moved to a locked position (FIGS. 6C and 9C). In the locked position, the fixing members 29, 30 of the first 25 and second 26 connector portions are engaged with each other such that the distal tube section 10a cannot rotate relative to the proximal tube section 10b. However, surgical procedures utilizing the surgical instrument 8 are not limited to the above disclosure. For example, the coupling can be moved to an unlocked position, rotated and relocked while being external to the patient.

A first connector portion may, for example, refer to the fixed sleeve and/or a portion of the rotating sleeve, and a second connector portion may, for example, refer to the sliding sleeve and/or a different portion of the rotating sleeve, or vice versa. Each of the first and second connector portions includes fixing members. One of the first and second connector portions is attached to the proximal tube section, and the other is attached to the distal tube section.

The illustrated exemplary embodiments of the surgical tool as set forth above are intended to be illustrative and not limiting. Various changes may be made without departing from the spirit and scope of the invention.

What is claimed is:

1. A surgical instrument comprising:
a distal tube section having a bend therein;
a proximal tube section; and
a coupling that couples the distal tube section to the proximal tube section, the coupling including (i) a first connector portion coupled to a first one of the distal tube section and the proximal tube section, and (ii) a second connector portion coupled to a second one of the distal tube section and the proximal tube section, the first and second connector portions being selectively engageable and disengageable with respect to each other,
the first and second connector portions each including selectively engageable fixing members such that the distal and proximal tube sections are rotationally fixed to each other when the fixing members of the second connector portion are engaged with the fixing members of the first connector portion, and the distal and proximal tube sections can rotate relative to each other when the fixing members of the second connector portion are disengaged from the fixing members of the first connector portion.

2. The surgical instrument of claim 1, wherein the coupling is coupled to a proximal end of the distal tube section, the distal tube section includes a cutting window at its distal end, and the bend is disposed between the distal and proximal ends of the distal tube section.

3. The surgical instrument of claim 1, wherein the proximal tube section includes a bend, the bend of the distal tube section and the bend of the proximal tube section can be disposed in a common plane or in different planes by rotating the first and second connector portions relative to each other.

4. The surgical instrument of claim 1, wherein:
the first connector portion includes a fixed sleeve;
the second connector portion includes a sliding sleeve, the sliding sleeve being movable toward and away from the fixed sleeve; and
the distal and proximal tube sections are rotationally fixed to each other when the sliding sleeve is moved toward the fixed sleeve such that the fixing members are engaged with each other, and the distal and proximal tube sections can rotate relative to each other when the sliding sleeve is moved away from the fixed sleeve such that the fixing members are disengaged from each other.

5. The surgical instrument of claim 4, wherein the coupling further comprises a rotating sleeve having a first end that is fixed to the distal tube section, and a second end that is rotatably coupled to the fixed sleeve.

6. The surgical instrument of claim 5, wherein the rotating sleeve includes a pin that extends into a slot of the sliding sleeve to prevent the rotating and sliding sleeves from rotating relative to each other.

7. The surgical instrument of claim 5, wherein the rotating sleeve is disposed inside of the fixed and sliding sleeves such that inner surfaces of the fixed and rotating sleeves, together with inner surfaces of the distal and proximal tube sections, form an internal passage of the surgical instrument that is maintained when the fixed and sliding sleeves are rotated relative to each other.

8. The surgical instrument of claim 5, further comprising a spring disposed between the rotating and sliding sleeves, the spring biasing the sliding sleeve toward the fixed sleeve such that the fixing members of the fixed and sliding sleeves engage with each other.

9. The surgical instrument of claim 4, wherein the coupling is coupled to a proximal end of the distal tube section, the distal tube section includes a cutting window at its distal end, and the bend of the distal tube section is disposed between the distal and proximal ends of the distal tube section.

10. The surgical instrument of claim 5, wherein the rotating sleeve is coupled to the proximal tube section and the sliding sleeve is coupled to the rotating sleeve.

11. A coupling for attaching tube sections of a surgical instrument to each other, the coupling comprising:
a fixed sleeve having first and second ends, the first end being fixedly attached to a first one of the tube sections;
a rotating sleeve having first and second ends, the first end being fixedly attached to a second one of the tube sections, the second end rotatably attached to the fixed sleeve;
a sliding sleeve disposed over an outer surface of the rotating sleeve and being slidable relative to the fixed and rotating sleeves between a locked position at which the sliding sleeve is closest to the fixed sleeve and an unlocked position at which the sliding sleeve is disposed away from the fixed sleeve; and
engageable fixing members provided on the second end of the fixed sleeve and on an end of the sliding sleeve that is engageable with the fixed sleeve,
wherein (i) when the sliding sleeve is moved to the locked position, the fixing members are engaged with each other to prevent relative rotation between the fixed and rotating sleeves, and (ii) when the sliding sleeve is moved to the unlocked position, the fixing members are disengaged from each other such that the fixed and rotating sleeves can rotate relative to each other.

12. The coupling of claim 11, wherein the second end of the rotating sleeve includes a flanged portion that is received within an accommodation portion at the second end of the fixed sleeve.

13. The coupling of claim 12, wherein the accommodation portion is configured to permit rotation and restrict axial movement of the rotating sleeve relative to the fixed sleeve.

14. The coupling of claim 11, further comprising:
a spring disposed between the rotating sleeve and the sliding sleeve, the spring biasing the sliding sleeve toward the locked position.

15. The coupling of claim 11, wherein the rotating sleeve includes a pin that extends into a slot of the sliding sleeve to prevent the rotating and sliding sleeves from rotating relative to each other.

16. The coupling of claim 11, wherein the rotating sleeve is disposed inside of at least part of the fixed and sliding sleeves such that the inner surfaces of the fixed and rotating sleeves, together with inner surfaces of the surgical instrument tube sections, form an internal passage of the surgical instrument that is maintained when the fixed and rotating sleeves are rotated relative to each other.

17. A method of performing a surgical procedure with a surgical instrument having (i) a distal tube section having a bend therein, (ii) a proximal tube section, and (iii) a coupling that couples the distal and proximal tube sections to each other, the method comprising:
inserting at least a portion of the distal tube section into a passage of a patient;
while the surgical instrument is inserted into the patient, moving the coupling to an unlocked position;
while in the unlocked position, rotating the distal and proximal tube sections relative to each other; and
after the rotating, moving the coupling to a locked position such that the distal and proximal tube sections cannot be rotated relative to each other.

18. The method of claim 17, wherein a fluid passage through an entirety of the surgical instrument is maintained intact while the coupling is in the unlocked position.

19. The method of claim 17, wherein the coupling includes (a) a fixed sleeve having first and second ends, the first end being fixedly attached to the proximal tube section, (b) a rotating sleeve having first and second ends, the first end being fixedly attached to the distal tube section, the second end rotatably attached to the fixed sleeve, (c) a sliding sleeve disposed over an outer surface of the rotating sleeve and being slidable relative to the fixed and rotating sleeves between the locked position at which the sliding sleeve is closest to the fixed sleeve and the unlocked position at which the sliding sleeve is disposed away from the fixed sleeve, and (d) engageable fixing members provided on the second end of the fixed sleeve and on an end of the sliding sleeve that is engageable with the fixed sleeve, wherein:
the coupling is moved to the unlocked position by sliding the sliding sleeve over the rotating sleeve and away from the fixed sleeve such that the fixing members on the fixed and sliding sleeves are disengaged from each other,
the rotating is performed by rotating the sliding and rotating sleeves relative to the fixed sleeve, and
the coupling is moved to the locked position by sliding the sliding sleeve over the rotating sleeve and toward the fixed sleeve such that the fixing members on the fixed and sliding sleeves are engaged with each other.

20. The method of claim 19, wherein the rotating sleeve is disposed inside of at least part of the fixed and sliding sleeves such that an inner surfaces of the fixed and rotating sleeves, together with inner surfaces of the distal and proximal tube sections, forms an internal passage of the surgical instrument that is maintained when the distal and proximal tube sections are rotated relative to each other.
